(19) United States
(12) Patent Application Publication
(10) Pub. No.: US 2025/0049612 A1
(43) Pub. Date: Feb. 13, 2025

(54) A DEVICE FOR OPENING AN EUSTACHIAN TUBE OF A PATIENT

(71) Applicant: EarFlo Inc., San Carlos, CA (US)

(72) Inventors: Matthew Peter Oldakowski, Applecross (AU); Intan Camellia Watono Oldakowska, Applecross (AU)

(21) Appl. No.: 18/719,255

(22) PCT Filed: Jun. 20, 2022
(86) PCT No.: PCT/AU2022/050620

§ 371 Date: Jun. 12, 2024

(30) Foreign Application Priority Data
Dec. 17, 2021 (AU) 2021904100

(51) Int. Cl. A61F 11/00

(52) U.S. Cl. CPC A61F 11/00

(57) ABSTRACT

The present disclosure provides a device for opening an eustachian tube of a patient. The device comprises a body having a top portion and an axis and a nose scaling portion positioned at the top portion of the body. The nose scaling portion has at least one opening and a sealing surface surrounding the at least one opening and arranged for contact with a nose of the patient. The device further comprises a mouthpiece positioned at the top portion of the body spaced apart from the nose sealing portion by a distance that at least approximates a distance between mouth and nose of the patient. The mouthpiece comprises a projection projecting from the top portion of the body in a direction approximately along the axis of the body. The projection providing a conduit for the fluid and being coupled to the source of the fluid. The projection comprises a first side portion that faces towards the nose sealing portion, the first side portion comprising a cut-away portion, at least one hole or perforations. The mouthpiece and the nose scaling portion are positioned and arranged such that, when the mouth of the patient encloses the cut-away portion, the at least one hole or the perforations at the first side portion, an opening of the nose of the patient is at least in close proximity of the sealing surface of the nose sealing portion whereby it is facilitated that pressurized air can penetrate from the source of the pressurized air into the nose of the patient to open an eustachian tube of the patient.

### FIELD OF THE INVENTION

The present invention relates to device for opening an eustachian tube of a patient.

### BACKGROUND OF THE INVENTION

Otitis media with effusion (OME) is a condition in which fluid collects in the middle ear of a patient and in which the Eustachian Tube (ET) does not open as it should during swallowing. The fluid can be present for a long period of time in some cases and OME may cause an infection.

OME may cause partial loss of hearing and is much more common in young children, such as children having an age between 1 and 3 years, than in older children or adults.

PCT international application PCT/US20/51744 discloses a systems and methods for opening the Eustachian Tubes. The present invention provides further improvement.

### SUMMARY OF THE INVENTION

In a first aspect of the present invention there is provided a device for opening an eustachian tube of a patient, the device comprising:
a body having a top portion and an axis;
a nose sealing portion positioned at the top portion of the body, the nose sealing portion having at least one opening and a sealing surface surrounding the at least one opening and arranged for contact with a nose of the patient; and
a mouthpiece positioned at the top portion of the body spaced apart from the nose sealing portion by a distance that at least approximates a distance between mouth and nose of the patient, the mouthpiece comprising a projection projecting from the top portion of the body in a direction approximately along the axis of the body, the projection providing a conduit for a fluid and being coupled to a source of the fluid, the projection comprising a first side portion that faces towards the nose sealing portion, the first side portion comprising a cut-away portion, at least one hole or perforations;
wherein the mouthpiece and the nose sealing portion are arranged such that, when the mouth of the patient encloses the cut-away portion, the at least one hole or the perforations, an opening of the nose of the patient is at least in close proximity of the sealing surface of the nose sealing portion whereby it is facilitated that pressurized air can penetrate from a source of the pressurized air into the nose of the patient to open an eustachian tube of the patient.

The mouthpiece and the nose sealing portion may be arranged such that, when the mouth of the patient encloses the cut-away portion, the at least one hole or the perforations at the first side portion, the sealing surface of the nose sealing portion forms a seal between an opening of the nose of the patient and the opening of the nose sealing portion whereby pressurized air can penetrate from the source of the pressurized air into the nose of the patient to open an eustachian tube of the patient.

The inventors have observed that, because of the projection (which may also be referred to as “spout”) of the mouthpiece has the first side portion with a cut-away portion, the at least one hole or the perforation, and which the mouth of the patient needs to enclose in order to suck the fluid in a comfortable manner, the patient needs to press the underside of the nose against the nose sealing portion which facilitates that the nose presses against the sealing surface of the nose sealing portion such that the sealing surface of the nose sealing portion forms the seal between the opening of the nose and the opening of the nose sealing portion.

The patient may be a child. In one specific embodiment the nose sealing portion and the mouthpiece are arranged such that, when a child having any facial anatomy drinks naturally and comfortably from the mouthpiece, the underside of the child's nose is pressed against the nose sealing portion with sufficient force to create a seal and generate intranasal pressure sufficient to open the eustachian tube without requiring excessive force which may be uncomfortable or impossible to achieve.

The device may further be arranged such that the child can learn how to use the device as the device is arranged such that a condition for comfortable drinking coincides with a condition in which sufficient pressure is applied to create a seal between the sealing surface of the nose sealing portion and the underside of the nose of the child sealing portion whereby the application of pressurized air into the nose of the patient to open an eustachian tube of the patient is possible.

The projection of the mouthpiece may comprise a base portion and a top portion. The base portion may have a substantially oval or rounded cross-sectional shape in a plane perpendicular to the axis of the body and may have a closed wall or walls surrounding the opening of the mouthpiece. Further, the projection may comprise an upper portion positioned over the base portion and which may comprise the first side portion having the cut-away portion, the at least one hole or the perforation.

In one specific embodiment the first side portion comprises a cut-away portion extending from an end-portion of the projection to the base portion. The cut-away portion of the projection, positioned over the base portion of the projection, may extend along 50-90% or more of a length of the projection. The cut-away portion of the first side portion may have a width (in a direction perpendicular to the axis of the body) that is 10, 20, 40, 60, 80, or even 100% of the width of the projection.

In one specific embodiment the cut-away portion has a width of 80% or more or even 100% of the width of the projection. The inventors have observed that if the patient's or the child's lips are close to the cut-away portion but not completely enclose the opening of the mouthpiece including the cutaway portion, they could entrain fluid by sucking in air at a high velocity. This resulted in slurping sounds and uncomfortable drinking (sucking in lots of air, straining to get fluid out). With the cut-away portion having a width of 80% or more or even 100% of the width of the projection, the child can only drink from the cup if the child's lips fully enclose the cut-away portion, avoiding the ability to slurp when there is a gap.

Further, the inventors have observed that, if the cut away portion is relatively wide, and the patient's face is not well positioned (the patient's mouth does not fully enclose the opening of the mouthpiece with the cut-way portion), the patient sucks air into the mouth through the cut-away portion. This provides feedback for the patient to move the head further down and apply more pressure on the nose sealing portion and allows the patient to learn how to use the device.

The device may further comprise a edge portion on which in use a chin of the patient rests. The edge portion is positioned at the top portion of the body. The edge portion is positioned such that the mouthpiece is located between the edge portion and the nose sealing portion. The edge portion, may provide a position at which the chin of the patients pivots when the face moves towards or away from the mouthpiece and nose sealing portion of the device. The edge portion, the mouthpiece and the nose sealing portion are positioned relative to each other such that, when the chin of the patient rests on the edge portion and the mouth of the patient encloses the opening of the projection of the mouthpiece including the first side portion, the sealing surface of the nose sealing portion forms a seal between an opening of the nose of the patient and the opening of the nose sealing portion.

The nose sealing portion may have a sealing surface that is moveable in a direction into the nose sealing portion upon application of a suitable pressure. For example, the nose sealing portion may comprise a material that is compressible and/or may comprise an air cushion, a pre-tension spring, or a material that has a compressible material property. Further, the nose sealing portion may be shaped such that the nose sealing portion bends under application of pressure wherein the bending causes a reaction force that is directed in a direction opposite to an applied compressive force. Alternatively, the nose sealing portion may be structured such that friction causes a reaction force that is directed in a direction opposite to an applied compressive force. The nose sealing portion may be arranged such that the reaction force, which opposes an applied compressive force (applied for example by the patient's nose pressing against the nose sealing portion), is at least largely constant when the applied compressive force moves the sealing surface of the nose sealing portion by 0-1 mm, 0-2 mm, 0-3 mm, 0-4 mm, 0-5 mm, 0-6 mm or more. The sealing surface of the nose sealing portion may be moveable by 0-2 mm, 0-4 mm, 0-6 mm or more when a nose of the patient pushes against the sealing surface of the nose sealing portion with a force of 0.1-1N, 1-4N, 4-8N, 8-12N, 12-16N.

In one specific embodiment the nose sealing portion is largely hollow and formed from a flexible polymeric material that may be rubber-like, whereby the nose sealing portion has a bellows-like spring function. The inventors have observed that the bellows-like spring function increases when the largely hollow nose sealing portion fills with air when compressed, which increases the reaction force and improves a seal between the nose of the patient and the nose sealing portion.

In this embodiment the nose sealing portion may be structured such that a reaction force which opposes an applied compressive force is largely constant when the applied compressive force moves the sealing surface of the nose sealing portion by 0-1 mm, 0-2 mm 0-3 mm, 0-4 mm or 0-5 mm.

The opening of the nose sealing portion may be a central opening which is surrounded by the nose sealing surface of the nose sealing portion. The opening may be of any shape, such as cylindrical shape with an axis parallel to the axis of the body of the device, but in one specific embodiment has a diameter that reduces in a direction from the sealing surface through a top wall portion of the nose sealing portion. The inventors have observed that the opening shaped this way has the advantage that the likelihood of erroneous blockage of the opening by the tip of the nose is reduced and an auditable noise caused by a motor positioned within the body and used for pressurising the air is reduced.

The source of the pressure may be adjustable whereby a pressure of air directed to the nose sealing portion is adjustable such that a pressure most suitable for a patient can be selected. Further, the device may comprise a pressure sensor for sensing a pressure of air directed to the nose sealing portion and electronic components forming a feedback loop with the source of the pressure whereby a desired pressure, which may be a pressure that is most comfortable for a patient but still able to open an eustachian tube of the patient, can be reached in an automated manner using the feedback loop.

The device may further be arranged such that two or more flow rates or air pressures can be selected. The device may also be arranged such that the flow rate or air pressure is variable or increases automatically during treatment from a relatively low value to a selected value. For example, the flow rate or air pressure could initially be relatively low so that the patient can then get used to the device and can then gradually increase to a desired value.

In another variation the device may be arranged such that a duration of pressurized air applied through the nose sealing portion to the patient can be selected. For example, a relatively short application of pressurized air may be selectable (such as in the order of 20-100 ms, for example approximately 50 ms) to minimise discomfort for the patient or alternatively a longer period of time for treatment may be selectable as required to optimize treatment. Further, the device may be arranged such that a number of uses of the device by the patient within a period of time, such as within a day, can be selected. For example, the device may be operable once a day and may then no longer be operable until the next day to ensure overtreatment doesn't occur.

The device comprises in another embodiment a pressure release valve that ensures that the pressurized air has during application not a pressure above a predetermined pressure. The device may further be arranged to generate an acoustic noise (such as a whistle or “duck call”) when the predetermined pressure is reached, by putting an acoustic element in series with the pressure release valve, for example after application of the pressure, which would provide a low-cost, real-time indication that the application of the pressure took place.

The device may also comprise an electric air pump arranged to generate the pressurized air. Further, the device may comprise a noise attenuating chamber positioned such that in use pressurized air generated by electrical pump transitions through the noise attenuation chamber before reaching the nose sealing portion, wherein the noise attenuation chamber is arranged to reduce an auditable acoustic noise that may be generated by the air pump.

In a second aspect of the present invention there is provided a drinking cup for facilitating middle ear health, the drinking cup comprising:
a body having a top portion and an axis;
a nose sealing portion positioned at the top portion of the body and having a nose sealing surface;
a mouthpiece positioned at the top portion of the body spaced apart from the nose sealing portion by a distance that at least approximates a distance between mouth and nose of the patient, the mouthpiece comprising a projection projecting from the top portion of the body in a direction approximately along the axis of the body, the projection providing a conduit for a fluid and being coupled to a source of the fluid and comprising a first side portion that faces towards the nose sealing portion, the first side portion comprising a cut-away portion, at least one hole or perforations;
wherein the mouthpiece and the nose sealing portion are arranged such that, when the mouth of the patient encloses the cut-away portion, the at least one hole or the perforations at the first side portion, the an opening of the nose of the patient is at least in the proximity of the nose sealing surface of the nose sealing portion whereby the likelihood that air can escape from the nose of the patient during drinking is at least reduced and a nasal pressurization that can open the eustachian tube is created.

The device in accordance with the second aspect of the present invention is typically arranged to block the nose of the patient during drinking. This may enable the patient to perform the toynbee maneuver and may be suitable for patients at less risk of eustachian tube dysfunction in order to maintain their middle ear health.

The invention will be more fully understood from the following description of specific embodiments of the invention. The description is provided with reference to the accompanying drawings.

### BRIEF DESCRIPTION OF THE DRAWINGS

FIG. 1 is an exploded perspective view of a device for opening an eustachian tube of a patient in accordance with an embodiment of the present invention;

FIG. 2 is a perspective view of an upper portion or lid of the device for opening an eustachian tube of a patient;

FIG. 3 is a top view of the upper portion or lid of the device for opening an eustachian tube of a patient;

FIG. 4 is cross-sectional view (section through plane A as shown in FIG. 3) of the device for opening an eustachian tube of a patient;

FIG. 5 is a cross-sectional view (section through plane B as shown in FIG. 3) of the device for opening an eustachian tube of a patient; and

FIG. 6 are views of components of the device for opening an eustachian tube of a patient in accordance with an embodiment of the present invention;

FIGS. 7 (a) and (b) are cross-sectional representations of components of the device for opening an eustachian tube of a patient in accordance with an embodiment of the present invention; and

FIG. 8 is a cross-sectional representation of a component of the device for opening an eustachian tube of a patient in accordance with an embodiment of the present invention.

### DETAILED DESCRIPTION OF SPECIFIC EMBODIMENTS

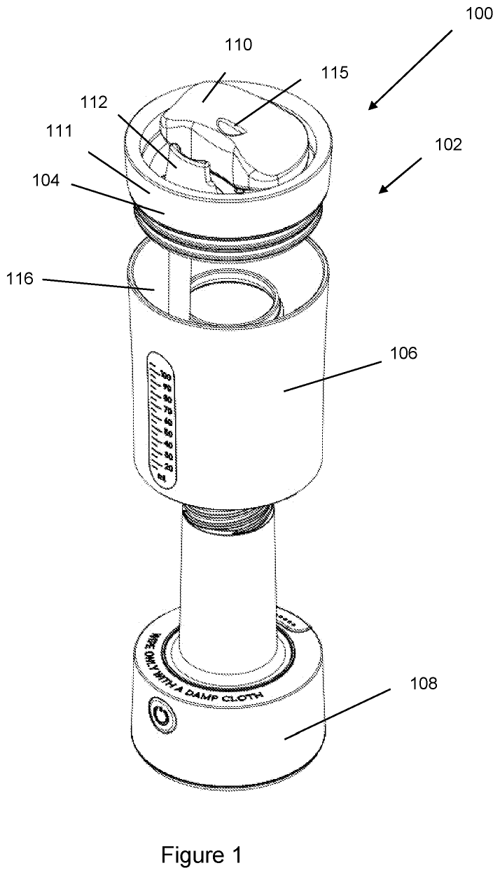

Referring initially to FIG. 1, a device for opening an eustachian tube of a patient in accordance with a specific embodiment of the present invention is now described. The device 100 is arranged to pressurize the nasal cavity of a patient during drinking in order to open the eustachian tube and ventilate the middle ear of a patient, such as a child.

The device 100 has a body 102 which comprises a top portion 104, a side portion 106 and a bottom/inner portion 108. The top portion 104 has a nose sealing portion 110, a mouthpiece 112 and an edge portion 111. The device 100 further comprises an electric air pump (not shown in FIG. 1) inside the bottom/inner portion 108 that can be coupled to a central opening 115 of the nose sealing portion 110. The device 100 also comprises a cavity 116 for a liquid coupled to the mouthpiece 112.

Edge portion 111 provides a position at which the chin of the patient pivots and the face moves towards or away from the top portion of the device 100. The edge portion 111, the mouthpiece 112 and the nose sealing portion 110 are positioned relative to each other such that, when the chin of the patient rests on the edge portion 111 and the mouth of the patient encloses the opening of the mouthpiece 112, a sealing surface 120 of the nose sealing portion forms a seal between an opening of the nose of the patient and the opening of the nose sealing portion 110 and pressurized air is directed into the nose of the patient through the opening in the nose sealing portion 115 to open the eustachian tube of a patient.

The body 102 of the device 100 comprises components formed from polymeric materials, which are fitted together using suitable fittings, such as press fittings. Portions of mating components may comprise a rubber material that facilitate a press-fit. Alternatively, the fittings may for example be threaded fittings.

The inventors have observed that if a force required to press the nose into the nose sealing portion for sealing is too significant and consequently the child cannot drink comfortably, the child may only drink smaller volumes or bolus (“sips”) of fluids at a time resulting in briefer swallowing times and lower swallowing force requirement, which trigger a lower eustachian tube muscle activation and affect eustachian tube opening. The device is arranged such that the child can learn how to use the device in a condition which is suitable for treatment and avoids the above-described situation. This is achieved by designing the device such that a condition for comfortable drinking the liquid coincides with a condition in which sufficient pressure is applied to create a seal between the sealing surface of the nose sealing portion and the underside of the nose of the child sealing portion, whereby the application of pressurized air into the nose of the patient to open an eustachian tube of the patient is possible. As the child will tend to use the device in the condition suitable for comfortable drinking, the child will then automatically use the device in a condition suitable for treatment.

The nose sealing portion and the mouthpiece are positioned and structured such that, when a child having any facial anatomy drinks naturally and comfortably from the mouthpiece, the underside of the child's nose is pressed against the nose sealing portion with sufficient force to create a seal and generate intranasal pressure sufficient to open the eustachian tube of the child without requiring excessive force which may be uncomfortable or impossible to achieve.

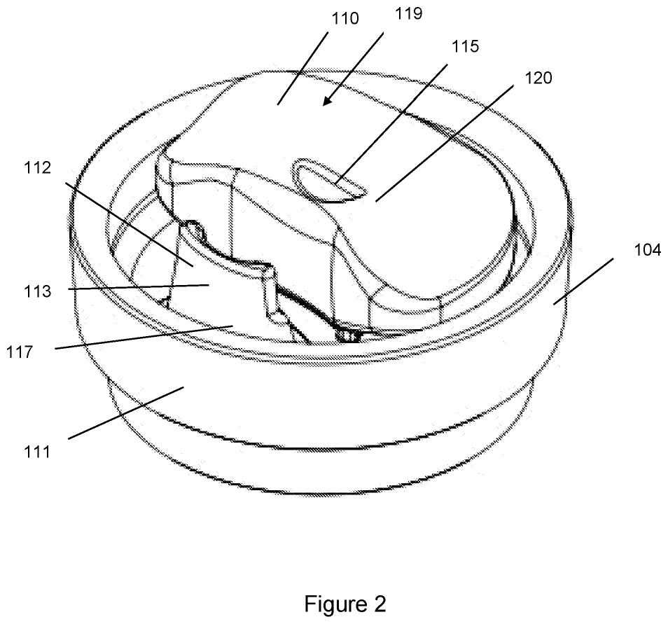

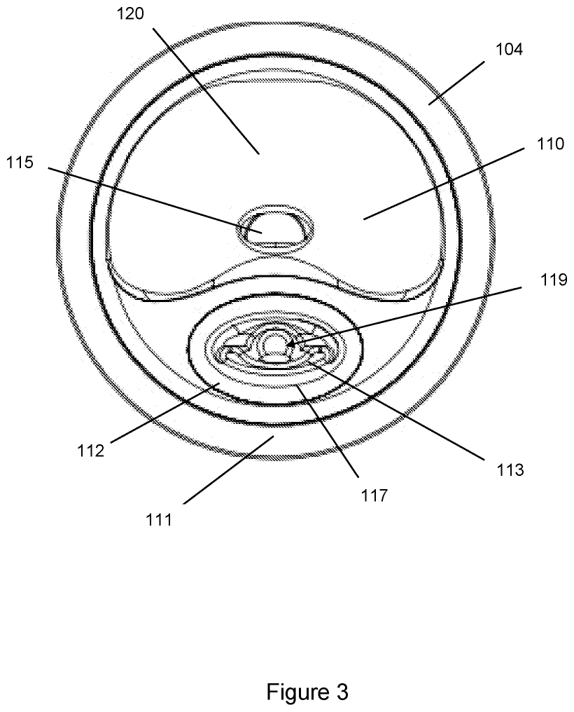

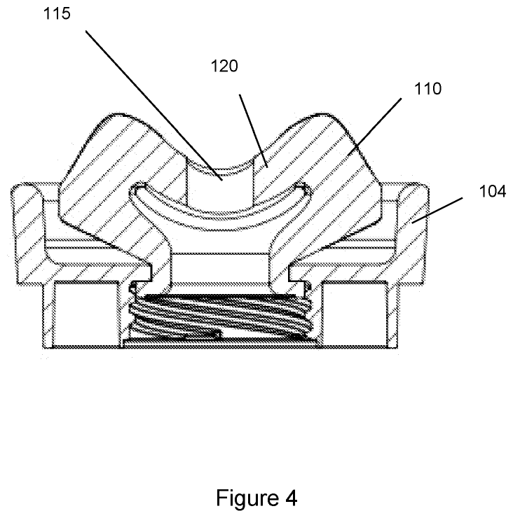

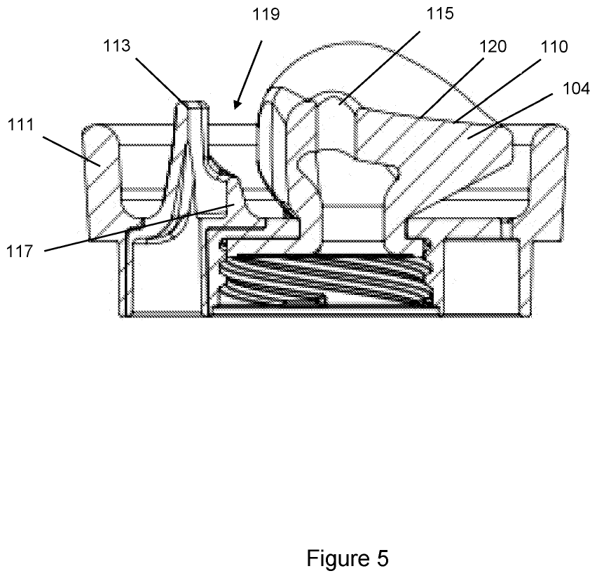

The top portion 104 including the nose sealing portion 110, mouthpiece 112 and the edge portion 111 will now be described in further detail with reference to FIGS. 2-7. Like components are given like reference numerals.

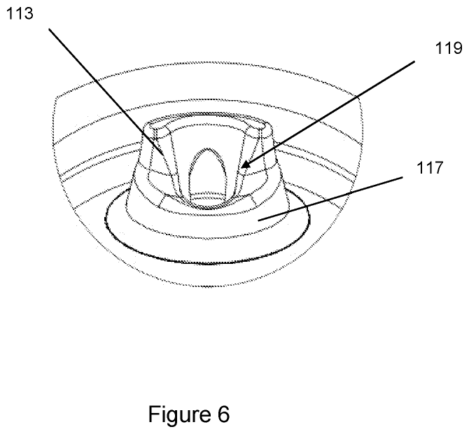

The mouthpiece 112 of the top portion 104 comprises a spout 113 which projects in a direction approximately along an axis of the body 102 of the device 100. The spout 113 has an opening which is coupled to a cavity for liquid 116 shown in FIG. 1. The spout 113 has an upper first side portion that faces towards the nose sealing portion 110 and has a cut-away portion 119, which may be best seen in FIG. 6. The mouthpiece 112 is arranged such that a mouth of the patient has to enclose the entire opening including the cut away portion 119 to suck the liquid through the mouthpiece form without sucking air.

As will be described further below in more detail, the nose sealing portion 110 is positioned such that the underside of the nose (outside the nostrils) of the patient presses against the nose sealing portion 110 when the mouth of the patient encloses the entire opening including the cut-away portion 119 of the spout 113, such that pressurized air can be directed through the opening 115 of the nose sealing portion 110 to open the eustachian tube of the patient.

The spout 113 has a base portion 117 above which the first side portion with the cut-away portion 119 is positioned. The base portion 117 has an oval cross-sectional shape in a plane perpendicular to the axis of the body 102 and has closed wall or walls surrounding the opening of the spout 113. The first side portion with the cut-away portion 119 is positioned over the base portion 117 and extends along 50-90% or more of a length of the spout 113. The cut-away portion 119 of the first side portion has a width (in a direction perpendicular to the axis of the body) that is 80 or even 100% of the width of the spout 113.

The nose sealing portion 110 is spaced from the spout 113 such that, when the mouth of the patient entirely encloses the opening of the spout 113 including the cut-away portion 119, the opening of the nose of the patients coincides with the opening 115 of the nose sealing portion 110. The nose sealing surface 120 is concavely shaped and arranged for contact with the nose of the patient. The concavely shaped sealing surface 120 is softer at an area in which the septum of the nose contacts the sealing surface 120, but is less soft on the outside of the nostrils of the patient in order to push up the edge of the nostrils for sealing when the nose of the patient pushes against the sealing surface 120, which allows comfort without sacrificing function.

The nose sealing portion 110 has a relatively large range (distance) over which the nose sealing portion 110 can be compressed under application of pressure by the nose of a patient and a reaction force is largely constant over a compression range of a few millimetres. The device 100 is designed such that the patient must apply some pressure to the nose sealing portion 110 to suck a fluid through the spout 113. This is achieved by the design of the spout 113 which, because of presence and location of the cut-away portion 119, requires pressing of the nose against the nose sealing portion 110 in order to be able to enclose the entire cut-away portion 119 with the mouth. As a consequence, the patient can't drink without receiving nasal pressurization therapy. In order to drink comfortably, it is required to press further against the nose sealing portion. As mentioned above, this is achieved by having a relatively long travel (distance over which the nose sealing portion 110 can be compressed) of the nose sealing portion 110. The nose sealing portion 110 may in another variation further comprise a tension spring element to facilitate providing largely constant compression. In addition, the nose sealing portion 110 may also comprise a tension ring (not shown) at a bottom portion to prevent tearing.

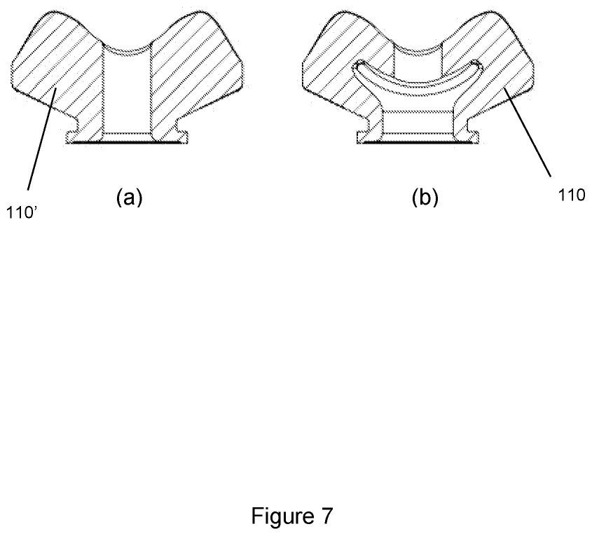

The nose sealing portion 110 may be composed of any suitable flexible material, such as a rubber-like polymeric material and may comprise a solid or hollow design. FIG. 7 (a) shows a nose sealing portion 110′ formed from flexible material which is solid around a central opening and FIG. 7 (b) shows a nose sealing portion 110 which has a hollow design. The hollow design has the advantage that the nose sealing portion has a bellows-like spring function. When the nose of the patient sealingly engages with the nose sealing portion 110 and compresses the nose sealing portion 110, a reaction force by the nose sealing portion 110 is initially largely constant, but increases again when the hollow nose sealing portion 110 fills with air, which improves a seal between the nose of the patient and the nose sealing portion 110.

The above-described hollow design of the nose sealing portion 110, formed from a relatively thin rubber-like material, has the advantage that the sealing surface 120 appears to be relatively soft, which is also a consequence of the hollow design. The sealing surface 120 is designed so that it is soft under the septum, but not as soft on the outside of the nostrils in order to push up the edge of the nostrils for sealing. This allows comfort without sacrificing function. When the nose of the patient sealingly engages with the hollow nose sealing portion 110 and compresses the nose sealing portion 110, a reaction force by the nose sealing portion 110 is initially largely constant, but increases again when the hollow nose sealing portion 110 fills with air, which improves a seal between the nose of the patient and the nose sealing portion 110.

Another advantageous feature of the hollow design of the nose sealing portion 110 is that the hollow nose sealing portion forms a reverberation chamber. Because the opening 115 at the top of the nose sealing portion 110 is chosen to be relatively small (such as 2-8 mm in diameter), most of the sound generated by a motor used for pressurizing the air is absorbed inside the hollow nose sealing portion 110. The opening of the of the nose sealing portion 110 is larger on top and smaller at the bottom, which reduced risk of blockage. A further advantage of the relatively small diameter of the opening 115 of the noise sealing portion 110 is that patients (in particular children) can't fit their fingers into the opening 115 in order to either tear the hole open, or erroneously block the hole tricking the device into thinking that a treatment has been delivered.

In this embodiment the nose sealing portion 110 is designed to have a largely constant reaction force in response to an applied force ranging from 10 to approximately 30N. In order to avoid that the nose sealing portion 110 can “bottom out” and artificially reduce this range, the nose sealing portion 110 is arranged to bottom out at the maximum a force 30N, which is a maximal force at which a patient (a child in this example) can drink comfortably.

The device 100 is further arranged such that a compression of less than 1 mm to 5 mm is sufficient to achieve sealing and a compression of 2-5 mm is sufficient to drink comfortably.

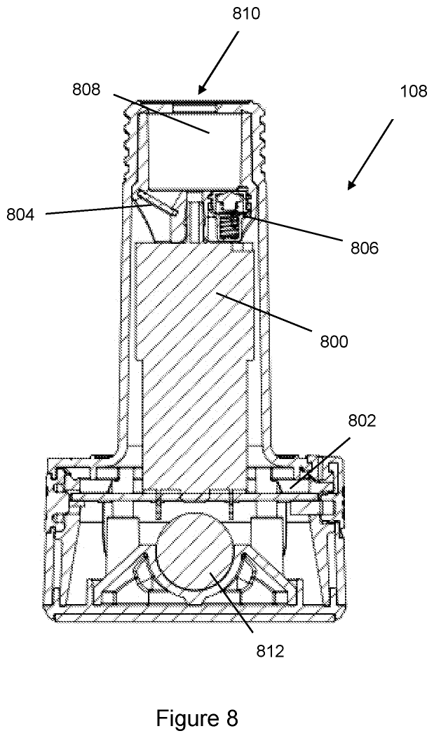

Referring now to FIG. 8, the bottom/inner portion 108, also shown in FIG. 1, is now described in further detail. The bottom/inner portion 108 includes a rubber mounted electric air pump 800, rubber mounted microelectronics 802, a pressure sensor 804, a pressure release valve 806, a sound attenuation chamber 808 coupled to an air outlet 810 and a battery 812. The sound attenuation chamber 808 is designed to attenuate noise, such as noise generated by the air pump 800 in order to minimize any discomfort by a person, such as a child, using the device 100. The rubber mounting of the air pump 800 and the microelectronics 802 reduces vibration that may be generated by the air pump 800 during use, which is also aimed at reducing discomfort by a person using the device 100.

The microelectronics 802 comprises in one embodiment a controller which controls the air pump 800 to adjust the pressurization of the nasal cavity such that a pressure most suitable for a patient can be selected. In this embodiment a feedback loop is formed comprising the controller, the air pump 800 and the air pressure sensor 804. The pressure sensor 804 detects a pressure when the user's nose is pressed against the nose sealing portion 110 and the controller receives a signal from the pressure sensor 804. The controller then controls the air pump 800 to either increase or decrease the air flow until a preselected air pressure, which may be a pressure that is most comfortable for a patient but can still open the eustachian tube, is reached. The preselected air pressure is selectable by a switch or dial or via an electronic device (not shown) which may for example be connected via bluetooth. The controller is also arranged such that two or more air flow rates can be chosen. Further, the device 100 has a setting in which the controller controls the air pump 800 to vary or increase the air flow rate from a relatively low value to a selected value so that the patient can initially get used to the device 100 and the pressure is the then gradually increased to a desired value.

The device 100 also has a setting in which the controller controls the air pump 800 such that a duration of pressurized air applied through the nose sealing portion to the patient can be selected. For example, a relatively short application of pressurized air, such as in the order of 50 ms, may be selectable to minimize discomfort.

In an alternative embodiment the device 100 does not comprise any or at least less electronic components for controlling operation of the device 100. In this embodiment the device 100 comprises a pressure release valve and ensures that the pressurized air does not have a pressure above a predetermined threshold pressure. In addition, the device 100 comprises a component (not shown) generating an acoustic noise (such as a whistle or “duck call”) when the predetermined threshold pressure indicating that the application of the required pressure takes place.

In one embodiment the device comprises a whistle and is arranged such that the acoustic noise is triggered whenever there is flow through the pressure release valve and through the whistle. The motor is kept running throughout and the pressure drop over the pressure release valve would be to a pre-set pressure (roughly 1 PSI). However, the swallow of the fluid by the child would only last 0.1-0.3 seconds. When the child stops swallowing (soft palate opens) then the air will escape down the back of the throat and the intranasal pressure will vent back to atmosphere.

In a further embodiment there is provided a drinking cup for facilitating middle ear health. The drinking cup does not comprise electric or electronic components and the nose sealing portion 110 does not have the opening 115, but is otherwise identical to the above-described device 100. The drinking cup is, in contrast to the device 100, not arranged to provide pressurized air, but is arranged for blocking the nose during drinking to allow nasal pressurization, which facilitates maintaining middle ear health.

A person skilled in the art will appreciate that variations of the described embodiments are possible. For example, the cut-away portion 119 may be replaced by a portion that has perforations or through-holes. Further, the nose sealing portion may not be hollow and may not have a bellows-like spring function, but may for example comprise a pre-tensioned spring, such as a constant force spring and/or may be solid (other than for a central opening). The nose sealing portion may also be arranged such that a spring function is provided by friction or a pressurized fluid such as air.

1. A device for opening an eustachian tube of a patient, the device comprising:
a body having a top portion and an axis;
a nose sealing portion positioned at the top portion of the body, the nose sealing portion having at least one opening and a sealing surface surrounding the at least one opening and arranged for contact with a nose of the patient; and
a mouthpiece positioned at the top portion of the body spaced apart from the nose sealing portion by a distance that at least approximates a distance between mouth and nose of the patient, the mouthpiece comprising a projection projecting from the top portion of the body in a direction approximately along the axis of the body, the projection providing a conduit for a fluid and being coupled to a source of the fluid, the projection comprising a first side portion that faces towards the nose sealing portion, the first side portion comprising a cut-away portion, at least one hole or perforations;
wherein the mouthpiece and the nose sealing portion are arranged such that, when the mouth of the patient encloses the cut-away portion, the at least one hole or the perforations at the first side portion, an opening of the nose of the patient is at least in close proximity of the sealing surface of the nose sealing portion whereby it is facilitated that pressurized air can penetrate from a source of the pressurized air into the nose of the patient to open an eustachian tube of the patient.

2. The device of claim 1 wherein the mouthpiece and the nose sealing portion are arranged such that, when the mouth of the patient encloses the cut-away portion, the at least one hole or the perforations at the first side portion, the sealing surface of the nose sealing portion forms a seal between an opening of the nose of the patient and the opening of the nose sealing portion whereby pressurized air can penetrate from the source of the pressurized air into the nose of the patient to open an eustachian tube of the patient.

3. The device of claim 1 wherein the patient is a child and the device is arranged such that a condition for comfortable drinking coincides with a condition in which sufficient pressure is applied create a seal between the sealing surface of the nose sealing portion and the underside of the nose of the child, whereby the application of pressurized air into the nose of the patient to open an eustachian tube of the patient is possible.

4. The device of claim 1 wherein the projection comprises the cut-away portion which extends along 50-90% or more of a length of the projection.

5. The device of claim 1 wherein the projection comprises the cut-away portion which has a width (in a direction perpendicular to the axis of the body) that is 10, 20, 40, 60, 80, or even 100% of the width of the projection.

6. The device of claim 1 further comprising an edge portion on which in use a chin of the patient rests, the edge portion being positioned at the top portion of the body and such that the mouthpiece is located between the edge portion and the nose sealing portion.

7. The device of claim 6 wherein the edge portion provides a position at which the chin of the patients pivots when the face moves towards or away from the mouthpiece and nose sealing portion of the device wherein the edge portion, the mouthpiece and the nose sealing portion are positioned relative to each other such that, when the chin of the patient rests on the edge portion and the mouth of the patient encloses the cut-away portion, the at least one hole or the perforations at the first side portion, it is facilitated that pressurized air can penetrate from a source of the pressurized air into the nose of the patient to open an eustachian tube of the patient.

8. The device of claim 1 wherein the nose sealing portion is shaped such that the nose sealing portion bends under application of pressure wherein the bending causes a reaction force that is directed in a direction opposite to an applied compressive force.

9. The device of claim 1 wherein the nose sealing portion has a sealing surface that is moveable in a direction into the nose sealing portion upon application of a suitable pressure.

10. The device of claim 8 wherein the nose sealing portion is arranged such that the reaction force, which opposes the applied compressive force, is at least largely constant when the applied compressive force moves the sealing surface of the nose sealing portion by 0-1 mm, 0-2 mm, 0-3 mm, 0-4 mm, 0-5 mm, 0-6 mm or more.

11. The device of claim 9 wherein the sealing surface of the nose sealing portion is moveable by 0-2 mm, 0-4 mm, 0-6 mm or more when a nose of the patient pushes against the sealing surface of the nose sealing portion with a force of 0.1-1N, 1-4N, 4-8N, 8-12N, 12-16N.

12. (canceled)

13. The device of claim 1 wherein the opening of the nose sealing portion has a diameter that reduces in a direction from the sealing surface through a top wall portion of the nose sealing portion.

14. The device of claim 1 wherein the source of the pressure is adjustable whereby a pressure of air directed to the nose sealing portion is adjustable such that a pressure most suitable for a patient can be selected.

15. The device of claim 1 further comprising a pressure sensor for sensing a pressure of air directed to the nose sealing portion and electronic components forming a feedback loop with the source of the pressurized air whereby a desired pressure, which may be a pressure that is most comfortable for a patient, can be reached in an automated manner using the feedback loop.

16. The device of claim 1 wherein the device is arranged such that two or more flow rates or air pressures can be selected.

17. The device of claim 1 wherein the device is arranged such that the flow rate or air pressure is variable or increases automatically during treatment from a relatively low value to a selected value.

18. The device of claim 1 wherein the device is arranged such that a duration of pressurized air applied through the nose sealing portion to the patient can be selected.

19. The device of claim 1 wherein the device is arranged such that a number of uses of the device by the patient within a period of time, such as within a day, can be selected.

20. The device of claim 1 comprising a pressure release valve that ensures that the pressurized air has during use of the device by the patient not a pressure above a predetermined pressure.

21-22. (canceled)

23. A drinking cup for facilitating middle ear health, the drinking cup comprising:
a body having a top portion and an axis;
a nose sealing portion positioned at the top portion of the body and having a nose sealing surface;
a mouthpiece positioned at the top portion of the body spaced apart from the nose sealing portion by a distance that at least approximates a distance between mouth and nose of the patient, the mouthpiece comprising a projection projecting from the top portion of the body in a direction approximately along the axis of the body, the projection providing a conduit for a fluid and being coupled to a source of the fluid and comprising a first side portion that faces towards the nose sealing portion, the first side portion comprising a cut-away portion, at least one hole or perforations;
wherein the mouthpiece and the nose sealing portion are arranged such that, when the mouth of the patient encloses the cut-away portion, the at least one hole or the perforations at the first side portion, the an opening of the nose of the patient is at least in the proximity of the nose sealing surface of the nose sealing portion whereby the likelihood that air can escape from the nose of the patient during drinking is at least reduced and a nasal pressurization that can open the eustachian tube is created.
